(19) United States
(12) Patent Application Publication
(10) Pub. No.: US 2015/0359489 A1
(43) Pub. Date: Dec. 17, 2015

(54) SMART MOBILE HEALTH MONITORING SYSTEM AND RELATED METHODS

(71) Applicant: VANDERBILT UNIVERSITY, Nashville, TN (US)

(72) Inventors: Franz Baudenbacher, Franklin, TN (US); Susan Eagle, Nashville, TN (US); Rene Harder, Nashville, TN (US); Jonathan Whitfield, Nashville, TN (US); Andre Diedrich, Nashville, TN (US); John B. Pietsch, Nashville, TN (US); Eric-Jan Manders, Atlanta, GA (US)

(21) Appl. No.: 14/762,909

(22) PCT Filed: Jan. 24, 2014
(86) PCT No.: PCT/US14/12977

§ 371 Date: Jul. 23, 2015

Related U.S. Application Data
(60) Provisional application No. 61/756,717, filed on Jan. 25, 2013.

(51) Int. Cl. A61B 5/00; A61B 5/11; G09B 5/00; A61B 5/04; G06F 19/00; G09B 19/00

(52) U.S. Cl. CPC A61B 5/6898; G06F 19/3406; G06F 19/3481; G09B 19/00; G09B 5/00; A61B 5/486; A61B 5/7282; A61B 5/0024; A61B 5/4836; A61B 5/742; A61B 5/04; A61B 5/1112; A61B 5/7455; A61B 2560/0475

(57) ABSTRACT

One aspect of the present disclosure is a smart patient monitoring system. A sensor is coupled to a patient and configured to detect biometric data associated with the patient. A mobile computing device includes a memory that stores computer-executable instructions and a processor executes the computer-executable instructions. The mobile computing device receives the biometric data from the sensor; processes the biometric data to monitor a health status of the patient; and provides therapeutic feedback related to the health status.

### RELATED APPLICATIONS

This application claims the benefit of U.S. Provisional Patent Application Ser. No. 61/756,717, filed Jan. 25, 2013, entitled “SMART PATIENT MONITORING SYSTEM.” The entirety of the provisional application is hereby incorporated by reference for all purposes.

### TECHNICAL FIELD

The present disclosure relates generally to health monitoring, and more particularly to a smart mobile health monitoring system and related methods of use.

### BACKGROUND

Traditional healthcare solutions focus on the treatment rather than the prevention of a disease. A steadily aging society with skyrocketing healthcare costs poses the need for a transformation from a reactive and hospital-driven healthcare system to a proactive, patient-centered and enabling healthcare system via medical equipment for home and ambulatory use. However, the medical equipment available for home and ambulatory use available today generally focuses on the pure acquisition of a single physiological parameter rather than multiple physiological parameters and treatment due to size, power and cost constraints.

### SUMMARY

In one aspect, the present disclosure includes mobile computing device. The mobile computing device includes a memory that stores computer-executable instructions and a processor that executes the computer-executable instructions. The execution of the computer-executable instructions enables the mobile computing device to receive biometric data detected by a sensor coupled to a body of a patient; process the biometric data to monitor a health status of the patient; and provide therapeutic feedback related to the current health status.

In another aspect, the present disclosure includes a system for smart mobile health monitoring that includes a sensor and a mobile computing device. The sensor is coupled to a patient and configured to detect biometric data associated with the patient. The mobile computing device includes a memory that stores computer-executable instructions and a processor that executes the computer-executable instructions. The execution of the computer-executable instructions allows the mobile computing device to at least receive the biometric data from the sensor; process the biometric data to monitor at least one of diagnose a medical condition of the patient, or diagnose a disease of the patient; and provide therapeutic feedback related to the health status and at least one of an activity of the patient and a body position of the patient.

In a further aspect, the present disclosure includes a non-transitory computer-readable device storing instructions executable by an associated processor to perform operations that facilitate smart mobile health monitoring. The operations include: receiving biometric data detected by a sensor coupled to a body of a patient; processing the biometric data to monitor a health status of the patient; providing therapeutic feedback related to the health status; and transmitting at least one of the biometric data, information related to the health status, and information related to the therapeutic feedback to an external device according to a wireless protocol.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a schematic illustration of an example smart mobile health monitoring system in accordance with an aspect of the present disclosure;

FIG. 2 is a schematic illustration of an example sensor configuration that can be utilized within the system of FIG. 1;

FIG. 3 is a schematic illustration of an example mobile computing device configuration that can be utilized within the system of FIG. 1;

FIG. 4 is a schematic illustration of an example external device configuration that can be utilized within the system of FIG. 1; and

FIG. 5 is schematic process flow diagram of an example method that facilitates health monitoring in accordance with an aspect of the present disclosure.

### DETAILED DESCRIPTION

The present invention generally relates to smart mobile health monitoring. Applications of smart mobile health monitoring include, but are not limited to: monitoring a health status while exercising, predicting and preventing falls, alerting emergency personnel of a change in health status, aiding in the diagnosis and management of patients with chronic conditions, and preventing and predicting medical events. The smart mobile health monitoring can be accomplished employing a sensor coupled to a patient and configured to detect biometric data (also referred to herein as “biomimetic data”) associated with the patient and a mobile computing device can receive the biometric data from the sensor (e.g., via a wired connection and/or a wireless connection); process the biometric data to at least one of monitor a health status of the patient, diagnose a medical condition of the patient, or diagnose a disease of the patient; and provide therapeutic feedback related to the health status. The therapeutic feedback can also be related to an activity of the patient and/or a body position of the patient. The mobile computing device can include a wireless transmitter that can transmit the biometric data, information related to the health status, or information related to the therapeutic feedback to an external device according to a wireless protocol.

As used herein, the term “patient” can refer to any warm-blooded organism including, but not limited to, human beings, pigs, rats, mice, dogs, goats, sheep, horses, monkeys, apes, rabbits, cattle, etc. When used herein, the term “health status” generally refers to a medical condition of a patient with respect to one or more properties represented by biometric data that can be detected by the sensor. The biometric data can include, but is not limited to: biopotential data, impedance data, biochemical data, temperature data, acoustical data, optical data, acceleration data, force data and pressure data.

The sensor can be auto-configurable and/or specialized for a particular patient. Examples of sensors that can be utilized within the smart sensor array include: biopotential sensors (e.g., to detect electrocardiogram (ECG), heart rate, etc.), impedance sensors (e.g., to detect hydration status, fluid shifts, respiration, cardiac output, etc.), acceleration sensors (e.g., to detect fall, activity, body position, etc.), pressure sensors (e.g., to detect diastolic blood pressure, mean blood pressure, systolic blood pressure, pulse pressure waveforms, etc.), and/or different types of sensors that can contribute to the smart health monitoring of the patient. The biopotential sensor can be a type of sensor that can detect electrocardiogram (ECG), skin potential (EDA), electroencephalogram (EEG), electromyogram (EMG), a heart rate, body impedance, a fluid status, a respiration, a cardiac output, a fall, an activity, a body position, a pulse wave form, blood oxygen levels, a respiratory CO₂ value, a plethysmograph signal, venous/arterial blood pressure waveform, diastolic blood pressure, a systolic blood pressure, or another biopotential that can be used in the monitoring of a health status, diagnosing a condition, and/or diagnosing a disease. The sensor is not limited to a single sensor; the sensor can include a plurality of individual sensors or electrodes. As an example, the sensor can be a configurable smart sensor array that can be coupled to the mobile computing device. The sensor can detect one or more parameters correlating to different medical conditions, including, but not limited to: arrhythmias, cardio vascular disease, myocardial infarction, heart failure, orthostatic hypotension, syncope, autism spectrum disorder, malnutrition, etc. The one or more parameters that are detected can indicate the health status of the patient.

The mobile computing device can communicate with the sensor and/or an external device according to a wireless protocol. Examples of mobile computing devices include, but are not limited to: smart phone devices, tablet computing devices, laptop computing device, personal media player devices, personal entertainment systems, or a device that includes at least a display, an input device, a wireless transceiver/hub, and a non-transitory computer readable medium storing executable instructions for a user interface, which can be used to display the biometric data, data derived from the biometric data, the information about the health status, or the information about the therapeutic feedback at the display and accept input from the user at the input device, as well as a processor configured to execute the stored instructions. The wireless transmitter of the mobile computing device can be used to accomplish the mobile monitoring of one or more medical parameters of the patient while the patient has the capability of movement and/or motion. The wireless transmitter generally refers to a transmitter that does not require a wired connection to transmit the data. The wireless transmitter can employ wireless body area network technologies, such as: Bluetooth (BT), Bluetooth low energy (BLE), ZigBee, ANT+, WiFi, etc. The mobile computing device can be capable of securely transmitting information via an appropriate encryption algorithm.

The following paragraphs include definitions of exemplary terms used within this disclosure. Except where noted otherwise, variants of all terms, including singular forms, plural forms, and other forms, fall within each exemplary term meaning. Except where noted otherwise, capitalized and non-capitalized forms of all terms fall within each meaning.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or figures unless specifically indicated otherwise.

In the context of the present disclosure, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof. As used herein, the term “and/or” can include any and all combinations of one or more of the associated listed items. “Or,” as used herein, except where noted otherwise, is inclusive, rather than exclusive. In other words, “or” is used to describe a list of alternative things in which one may choose one option or any combination of alternative options. For example, “A or B” means “A or B or both” and “A, B, or C” means “A, B, or C, in any combination or permutation.” If “or” is used to indicate an exclusive choice of alternatives or if there is any limitation on combinations of alternatives, the list of alternatives specifically indicates that choices are exclusive or that certain combinations are not included. For example, “A or B, but not both” is used to indicate use of an exclusive “or” condition. Similarly, “A, B, or C, but no combinations” and “A, B, or C, but not the combination of A, B, and C” are examples where certain combinations of alternatives are not included in the choices associated with the list.

The present disclosure includes reference to block diagrams and/or flowchart illustrations of methods, apparatus (systems) and/or computer program products according to certain aspects of the disclosure. It is understood that each block of the block diagrams and/or flowchart illustrations, and combinations of blocks in the block diagrams and/or flowchart illustrations, can be implemented by computer program instructions. These computer program instructions may be provided to a processor of a general purpose computer, special purpose computer, and/or other programmable data processing apparatus to produce a machine, such that the instructions, which execute via the processor of the computer and/or other programmable data processing apparatus, create means for implementing the functions/acts specified in the block diagrams and/or flowchart block or blocks.

These computer program instructions may also be stored in a computer-readable memory that can direct a computer or other programmable data processing apparatus to function in a particular manner, such that the instructions stored in the computer-readable memory produce an article of manufacture including instructions, which implement the function/act specified in the block diagrams and/or flowchart block or blocks.

The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions/acts specified in the block diagrams and/or flowchart block or blocks.

Accordingly, the present disclosure may be embodied in hardware and/or in software (including firmware, resident software, micro-code, etc.). Furthermore, aspects of the present disclosure may take the form of a computer program product on a computer-usable or computer-readable storage medium having computer-usable or computer-readable program code embodied in the medium for use by or in connection with an instruction execution system. A computer-usable or computer-readable medium may be any non-transitory medium that can contain or store the program for use by or in connection with the instruction or execution of a system, apparatus, or device.

The computer-usable or computer-readable medium may be, for example but not limited to, an electronic, magnetic, optical, electromagnetic, infrared, or semiconductor system, apparatus or device. More specific examples (a non-exhaustive list) of the computer-readable medium can include the following: a portable computer diskette; a random access memory; a read-only memory; an erasable programmable read-only memory (or Flash memory); and a portable compact disc read-only memory.

“Operative communication,” as used herein includes, but is not limited to, a communicative relationship between devices, logic, or circuits, including wired and wireless relationships. Direct and indirect electrical, electromagnetic, and optical connections are examples of connections that facilitate operative communications. Two devices are in operative communication if an action from one causes an effect in the other, regardless of whether the action is modified by some other device. For example, two devices in operable communication may be separated by one or more of the following: i) amplifiers, ii) filters, iii) transformers, iv) optical isolators, v) digital or analog buffers, vi) analog integrators, vii) other electronic circuitry, viii) fiber optic transceivers, ix) Bluetooth communications links, x) IEEE 802.11 communications links, xi) satellite communication links, xii) gateways, repeaters, routers, and hubs, xiii) wired or wireless networks, xiv) mobile communications towers, and xv) other wired or wireless communication links. Operative communication may be facilitated by and exist between devices using, for example, the internet or service provider networks. As another example, an electromagnetic sensor is in operative communication with a signal if it receives electromagnetic radiation from the signal. As a final example, two devices not directly connected to each other, but both capable of interfacing with a third device, e.g., a central processing unit (CPU), are in operative communication.

“Processor,” as used herein includes, but is not limited to, one or more of virtually any number of processor systems or stand-alone processors, such as microprocessors, microcontrollers, central processing units (CPUs), distributed processors, paired processors, and digital signal processors (DSPs), in any combination. The processor may be associated with various other circuits that support operation of the processor, such as random access memory (RAM), read-only memory (ROM), programmable read-only memory (PROM), erasable programmable read-only memory (EPROM), clocks, decoders, memory controllers, or interrupt controllers, etc. These support circuits may be internal or external to the processor or its associated electronic packaging. The support circuits are in operative communication with the processor. The support circuits are not necessarily shown separate from the processor in block diagrams or other drawings.

“Software,” as used herein includes a set of computer readable or executable instructions stored on a non-transitory computer readable medium that can be executed to cause a computer or another electronic device to perform functions, actions, or behave in a desired manner. The instructions may be embodied in various forms such as routines, algorithms, modules or programs including separate applications or code from dynamically linked libraries. Software may also be implemented in various forms such as a stand-alone program, a function call, a servlet, an applet, instructions stored in a memory, part of an operating system, or other types of executable instructions. It will be appreciated by one of ordinary skill in the art that the form of software is dependent on, for example, requirements of a desired application, the environment it runs on, or the desires of a designer/programmer or the like. Software may be embodied as an “application.”

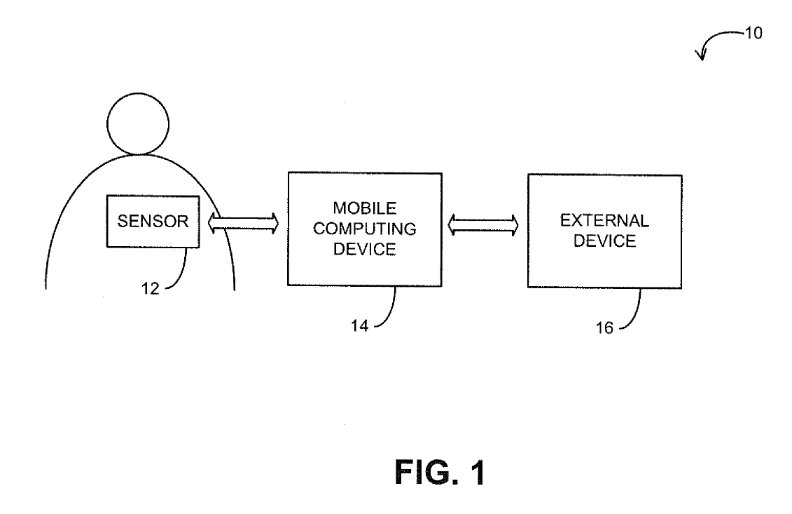

Referring now to FIG. 1, illustrated is a schematic illustration of an example smart mobile health monitoring system 10 in accordance with an aspect of the present disclosure. The smart mobile health monitoring system includes a sensor 12 associated with (e.g., coupled to, in proximity with, attached to, etc.) a patient in a manner that allows the sensor to detect biometric data from the patient. The sensor 12 is coupled to a mobile computing device 14 via a wired connection or a wireless connection (employing a wireless protocol) for transmission of the biometric data from the sensor to the mobile computing device. The mobile computing device 14 can receive the biometric data from the sensor 12 and process the biometric data to monitor a health status of the patient, diagnose a medical condition of the patient, and/or diagnose a disease of the patient. Additionally, the mobile computing device 14 can provide therapeutic feedback related to the health status of the patient. The therapeutic feedback can also be related to an activity of the patient and/or a body position of the patient.

The mobile computing device 14 can provide the therapeutic feedback to the patient (e.g., by a display, an alarm, a speech, or another type of alert). In response to the therapeutic feedback, the mobile computing device 14 can receive speech input or other type of input (e.g., from the patient and/or a person administering treatment to the patient). The input can include, but is not limited to, information about an activity, symptoms, status, a medication, a body position and/pr food consumption. As an example, the mobile computing device 14 can alert an external device 16 to take an action (e.g., initiate a treatment procedure and/or a preventive procedure) in response to the speech input. However, the speech input is not required for the external device 16 to take the action.

The mobile computing device 14 can be coupled to the external device 16 (e.g., via a wired connection or a wireless connection employing a wireless protocol) to transmit the biometric data, information regarding the health status, or information regarding the therapeutic feedback to the external device. In response, the external device 16 can provide an input to the mobile computing device 14 and/or the sensor 12 that can include a query, processed data, an instruction to adjust at least one of the health status and the therapeutic feedback for diagnostic purposes, and/or an instruction to change patient treatment regiments.

The external device 16 can be a therapeutic device configured to deliver a therapeutic treatment to the patient based on the information received from the mobile computing device 14. The external device 16 can access or include a secured external data store that can be accessible to a physician, other authorized medical personnel or caregiver associated with the patient. The external device 16 can include an expert system that can, for example, determine a procedure that can be used on the patient based on the information received from the mobile computing device 14.

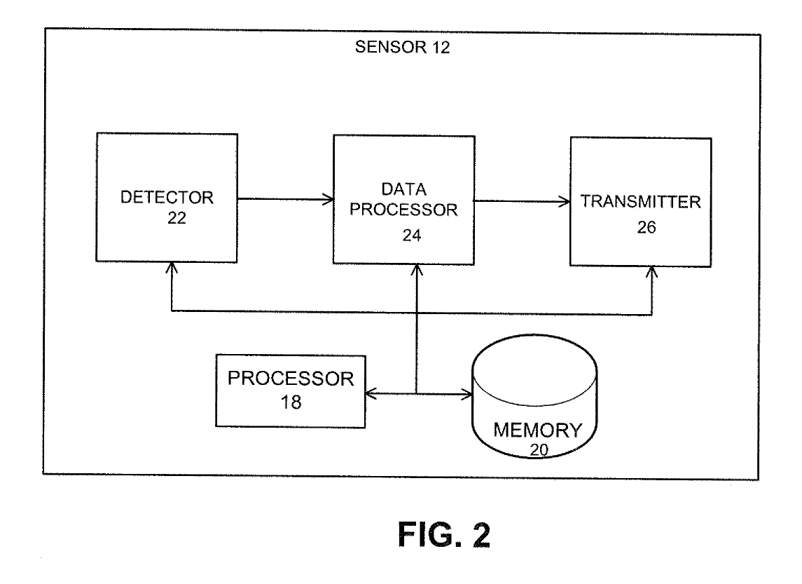

FIG. 2 shows a schematic diagram of a sensor 12 that can be utilized within the smart mobile heath monitoring system. Although a single sensor is illustrated, the sensor 12 can be understood to include a plurality of sensors, each receiving inputs that contribute to the biometric parameter. The sensor 12 can be remotely configured, dynamically configured, and/or adapted for a specific physiological state, a condition, and/or a specific disease to provide optimized feedback and/or diagnostic capabilities. The sensor 12 can include a detector 22 that can detect the biometric parameter, a data processor 24 that can process the detected biometric parameter (e.g., transform the biometric parameter into signal and/or a data type that can be received by the mobile computing device), and a transmitter 26 that can transmit the processed biometric parameter to the mobile computing device (e.g., via a wired and/or a wireless interface with the mobile computing device). The detector 22, the data processor 24, and the transmitter 26 can be referred to collectively as “the components of the sensor”.

One or more of the components of the sensor can be implemented by computer program instructions that can be stored in memory 20, a non-transitory computer-readable memory (e.g., an electronic, magnetic, optical, electromagnetic, infrared, or semiconductor system, apparatus or device) and provided to a processor 18 (e.g., microprocessor, and/or other programmable data processing apparatus). The processor 18 can execute the instructions such that the sensor can implement the functions of one or more of the components of the sensor. In an example, the memory 20 can be based on a memory card (e.g., a SD card) and the processor 18 can be based on a microcontroller (e.g., an Atmel xMega microcontroller). The sensor 12 can include a power source (e.g., one or more batteries or the like) that can power one or more of the components of the sensor.

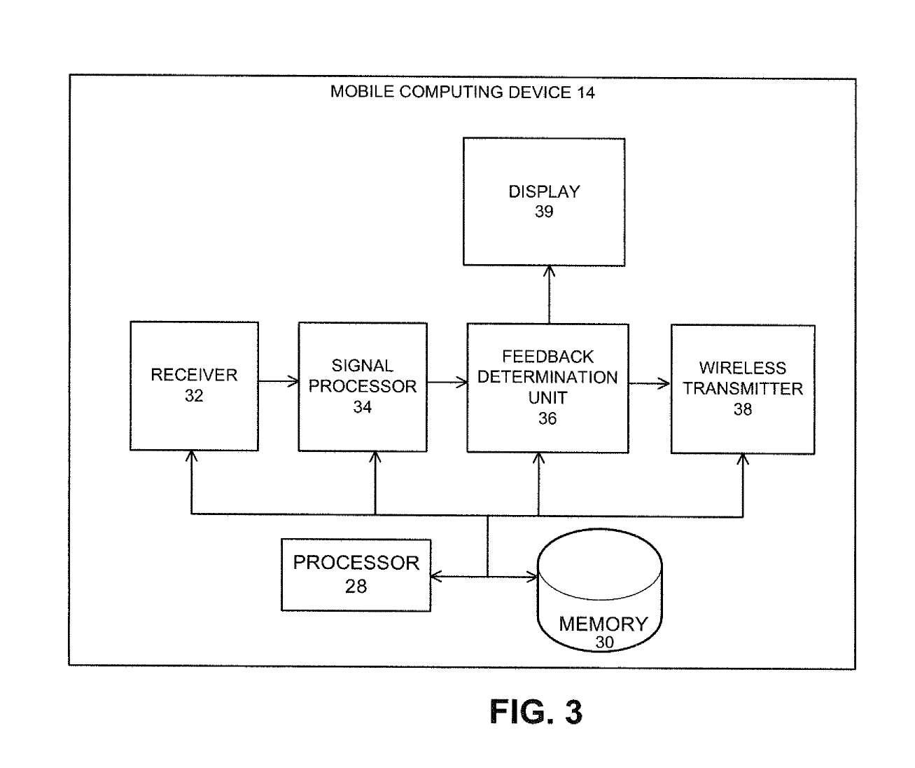

FIG. 3 shows a schematic diagram of the mobile computing device 14 that can be utilized within the smart mobile heath monitoring system. The mobile computing device 14 can include a receiver 32 that can receive the biometric data (or a signal that includes the biometric data) from the sensor (e.g., transmitted across a wired connection or a wireless connection). The mobile computing device 14 can also include a signal processor 34 that can process the biometric data (or the signal including the biometric data) and determine a health status of the patient based on the biometric data. Based on the biometric data and/or the health status, a therapeutic feedback determination unit 36 can provide therapeutic feedback related to the health status. The therapeutic feedback (the biometric data and/or the health status) can be presented to a user on a display 39 of the mobile computing device 14. The mobile computing device 14 also includes a wireless transmitter that can transmit the biometric data, information regarding the health status, and/or information regarding the therapeutic feedback to an external device. The receiver 32, the signal processor 34, the therapeutic feedback determination unit 36, the wireless transmitter 38 and the display 39 can be referred to collectively as “the components of the mobile computing device.”

One or more of the components of the mobile computing device can be implemented by computer program instructions that can be stored in memory 30, a non-transitory computer-readable memory (e.g., an electronic, magnetic, optical, electromagnetic, infrared, or semiconductor system, apparatus or device) and provided to a processor 28 (e.g., microprocessor, and/or other programmable data processing apparatus). The processor 28 can execute the instructions such that the mobile computing device can implement the functions of one or more of the components of the mobile computing device.

The mobile computing device 14 can include a global positioning system (GPS) that can determine the location of the patient. The location can be transmitted to the external device 16 in connection with the biometric data, the information related to the health status, and/or the information related to the therapeutic feedback. The mobile computing device 14 can choose an external device 16 (or devices) to receive the biometric data, the information related to the health status, and/or the information related to the therapeutic feedback based on the location. For example, when a patient is experiencing a medical emergency, the mobile computing device 14 can send the biometric data, the information related to the health status, and/or the information related to the therapeutic feedback to an external device 16 (e.g., associated with a first responder or a hospital) in closest proximity to the location.

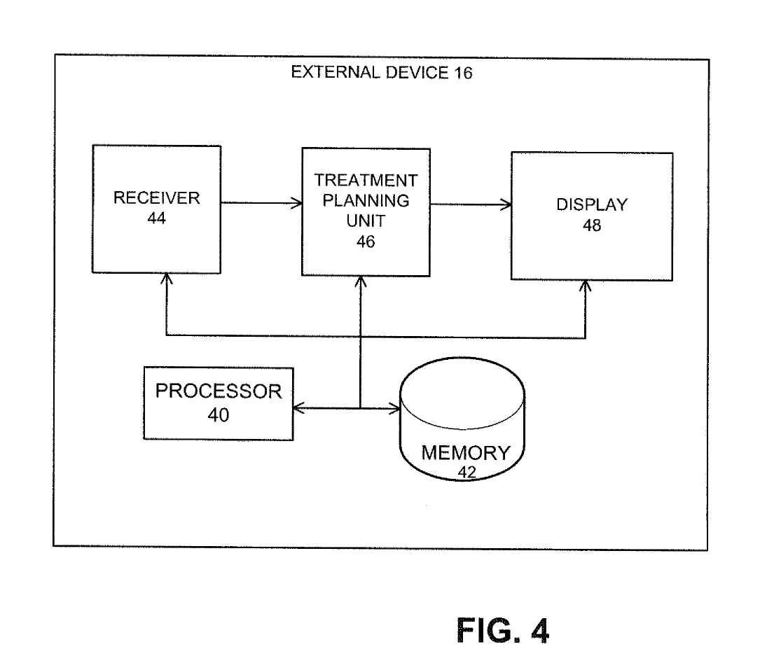

FIG. 4 shows a schematic diagram of an external device that can receive the biometric data, data derived from the biometric data, the information about the health status of the patient and/or the information about the therapeutic feedback from the mobile computing device. The external device can include a receiver 44 that can receive the biometric data, data derived from the biometric data, the data derived from the biometric data, the information about the health status of the patient and/or the information about the therapeutic feedback from the mobile computing device in a wireless transmission. Upon receiving the wireless transmission, a treatment planning unit 46 can determine a treatment for the patient based on the received biometric data, data derived from the biometric data, information about the health status of the patient and/or information about the therapeutic feedback from the mobile computing device. The treatment plan can be displayed on a display 48 and/or executed by a treatment unit. The biometric data, the data derived from the biometric data, the information about the health status of the patient, the information about the therapeutic feedback from the mobile computing device and/or the treatment plan can be stored in a secure data store (e.g., external to the external device 16 or internal to memory 42) that is accessible to a physician, caregiver and/or other authorized medical personnel associated with the patient. The receiver 44, the treatment planning unit 46, and the display 48 can be referred to collectively as “the components of the external device.”

One or more of the components of the external device can be implemented by computer program instructions that can be stored in memory 42, a non-transitory computer-readable memory (e.g., an electronic, magnetic, optical, electromagnetic, infrared, or semiconductor system, apparatus or device) and provided to a processor 40 (e.g., microprocessor, and/or other programmable data processing apparatus). The processor 40 can execute the instructions such that the external device can implement the functions of one or more of the components of the external device.

The external device 16 can be coupled to one or more additional devices to implement the treatment plan. Examples of additional devices include, but are not limited to: defibrillators, sphygmomanometers, accelerometers, pulse oximeters, blood pressure measurement systems, drug, blood or fluid infusion devices, and respirators. The additional devices can communicate wirelessly with the mobile computing device 14 to provide additional biometric parameters and treatment/responses for the patient. The external device 16 (e.g., associated with a physician, caregiver, hospital, an expert system, or the like) can use this data from the additional devices to monitor the treatment plan and/or to initiate a new treatment plan. The mobile computing device 14 can be electrically shielded from the actions of the additional devices. Additionally, any wired connection to the sensor 12 and/or the additional devices can also be electrically shielded. The sensor 12 and/or the mobile device 14 can be operated by an internal battery (e.g., the battery can be chargeable via a radio frequency (RF) charging circuit or another type of non-contact charging circuit).

As an example, the smart mobile health monitoring system 10 of FIG. 1 can be wireless with a miniature, cost-effective, and/or wearable sensor 12. The smart mobile health monitoring system 10 can be personalized to meet clinical and/or personal needs of the patient (e.g., the sensor 12 can be configured with times to sense the biometric parameter). In one example, the smart mobile health monitor can be a smart ECG device that can record an ECG. The sensor 12 can be a twelve-lead ECG (e.g., the leads can be electrically shielded) that can send the biometric data to a mobile computing device 14 associated with a paramedic or other first responder or on-scene caregiver, which can transmit the data from the emergency setting to the external device 16 to display the ECG data in a manner familiar to a physician (e.g., on a dynamic twelve-lead ECG grid that scales dynamically with a zoom level of the ECG with a dynamically adjustable grid density).

The external device 16 can be associated with the nearest hospital with a qualified interventional cardiology team and/or a physician associated with the patient to initiate pre-hospital thrombolytic therapy and/or fast track the treatment of patients with myocardial infarct and reduce the time between diagnosis and treatment. Immediate transmission of a paramedic performed recording to a qualified infarct team allows for effective triage of patients with ST-elevation myocardial infarction (STEMI), reduces the time to balloon angioplasty, and have the potential to minimize the degree of myocardial damage and loss.

The external device 16 can include a tracking application that estimates or allows the physician to estimate the location and arrival time of patient at hospital to allow for advanced planning of any potential intervention. The patient's care in transit to the hospital can be supervised effectively by the physician, and any necessary medical interventions can be provided immediately or upon the patient's arrival at the hospital, increasing the likelihood of a positive medical outcome. The external device 16 can also allows the physician to provide instant therapeutic feedback (e.g., selected among predefined message templates) to the paramedic or other first responder after reviewing the ECG data to manage the patient's care en route to the hospital.

This smart mobile health monitoring system 10 (also referred to as a smart ECG device) can overcome current obstacles, including current non-universal ECG transmission and costly alterations of hospital infrastructure in order to receive ECGs. The miniature, battery operated, wireless Smart ECG device communicates with smart phones, allowing the ECG to be recorded transmitted to the nearest hospital with interventional cardiology abilities by pressing a single button. A mobile device application allows a physician to retrieve the ECG data from an online service of the hospital and display the ECG data on a physician familiar standard grid. Instant therapeutic feedback can be provided to the paramedics via a secure messaging system embedded into a user interface of the mobile device application. The form factor of the ECG device is small and fits easily into the pocket of a doctor or paramedic's coat. The system is designed to be compatible with existing data infrastructure, and can be directly integrated in existing patient database systems.

The smart mobile health monitoring system 10 of FIG. 1 can be used to monitor autonomic function during sleep in autistic patients. For example, the sensor 12 can be used to measure activity, electrodermal activity, and polysomnography of the autistic patient to study the heart rate of autistic patients during sleep. Information related to the heart rate during sleep can be sent to mobile computing device 14 and then aggregated at an external device 16 associated with a medical study.

The smart mobile health monitoring system 10 of FIG. 1 can also be used to detect and predict syncope by sensing biometric parameters associated with a patient and analyzing the biometric parameters on the mobile computing device 14. The external device 16 can be used to perform an action to prevent the syncope upon receiving a signal from the mobile computing device 14. For example, when indicated by the health status exceeding a threshold indicating that syncope may occur, communicating to an external electrical stimulator to provide electrical stimuli to the patient to prevent the syncope. The smart mobile health monitoring system 10 can also be used to detect and monitor malnutrition levels in children (e.g., through an application on the mobile computing device 14 and/or the external device 16 in connection with one or more impedance sensors).

For example, the malnutrition of children in developing countries or rural locations within developed countries can be managed by sensing and processing an impedance parameter (e.g., based on a bioelectrical impedance algorithm) and sending the parameter and/or the processed parameter to an external device (e.g., associated with a hospital, doctor, researcher, or the like within the developed country and/or within a developed country). The bioelectrical impedance analysis (BIA) can rely on change in impedance of electrical current traveling through the body and an analytical approach based on this measurement. Bioelectrical Impedance Vector analysis is an example of the analytical approach that uses a graphical technique to determine body composition by plotting changes in total body water and cell membrane functionality. Coupling BIVA and decision support and longitudinal record keeping can further guide a nutritional intervention and facilitates tracking a patient through an episode of care.

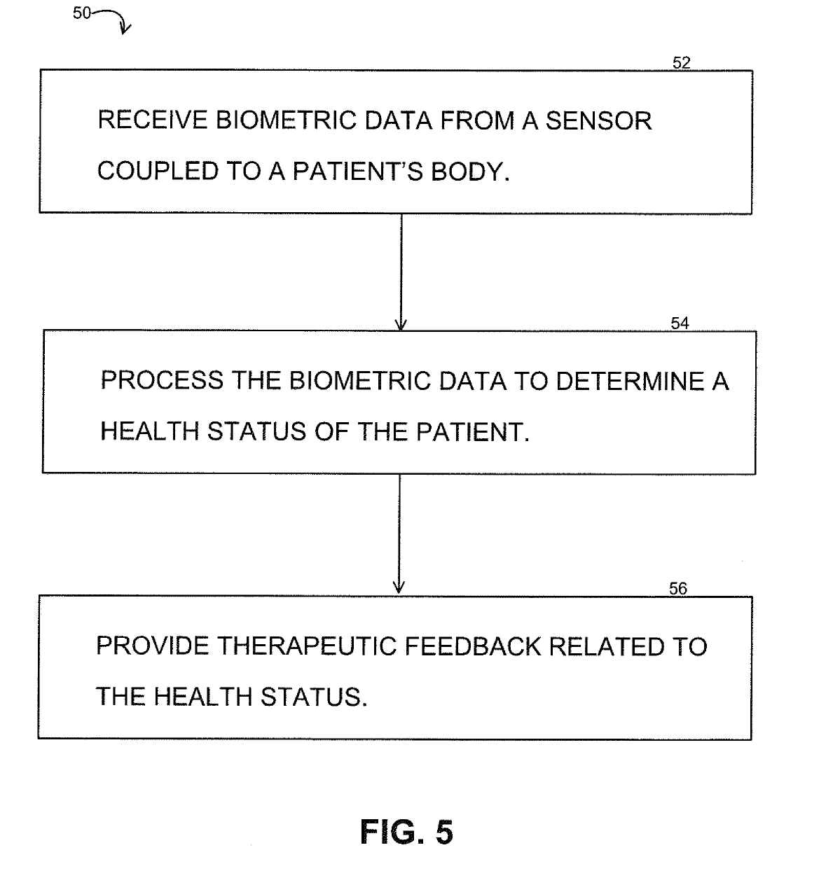

In view of the foregoing structural and functional features described above, a method in accordance with various aspects of the present invention will be better appreciated with reference to FIG. 5. While, for purposes of simplicity of explanation, the method of FIG. 5 is shown and described as executing serially, it is to be understood and appreciated that the present invention is not limited by the illustrated order, as some aspects could, in accordance with the present invention, occur in different orders and/or concurrently with other aspects from that shown and described herein. Moreover, not all illustrated features may be required to implement a methodology in accordance with an aspect of the present invention. It will be appreciated that some or all of each of these methods can be implemented as machine-executable instructions stored on a non-transitory computer readable device (e.g., memory 20, 30 and/or 42). The instructions can be executed by a processor (e.g., processor 18, 28 and/or 40) to facilitate the performance of operations of the method.

FIG. 5 illustrates an example of a method that facilitates health monitoring. At 52, biometric data (e.g., related to an ECG, a heart rate, a hydration status, fluid shift, a respiration, a cardiac output, a fall, an activity, a body position, a diastolic blood pressure, mean blood pressure, a systolic blood pressure, etc.) can be received (e.g., at mobile computing device 14 across a wired or a wireless connection employing a wireless protocol, such as: a Bluetooth protocol, a Bluetooth low energy protocol, a ZigBee protocol, an ANT+ protocol, a WiFi protocol, etc.) from a sensor (e.g., sensor 12) coupled to a patient's body. At 54, the biometric data is processed (e.g., by the mobile computing device 14) to determine a health status of the patient. At 56, therapeutic feedback (e.g., to the patient and/or to an external device) related to the health status can be provided (e.g., by the mobile computing device 14).

From the above description, those skilled in the art will perceive improvements, changes and modifications. Such improvements, changes, and modifications are within the skill of one in the art and are intended to be covered by the appended claims. All references cited herein and listed above are incorporated by reference in their entireties as needed and as discussed herein.

The following is claimed:

1. A mobile computing device comprising:
a memory that stores computer-executable instructions; and
a processor that executes the computer-executable instructions to at least:
receive biometric data detected by a sensor coupled to a body of a patient;
process the biometric data to monitor a health status of the patient; and
provide therapeutic feedback related to a current health status of the monitored health status.

2. The mobile computing device of claim 1, wherein the biometric data comprises at least one of biopotential data, impedance data, biochemical data, temperature data, acoustical data, optical data, acceleration data, force data and pressure data.

3. The mobile computing device of claim 1, wherein the processor executes the computer-executable instructions to transmit information regarding the health status to an external device.

4. The mobile computing device of claim 3, wherein the external device comprises a therapeutic device configured to follow at least one of a treatment procedure and a preventive procedure for the patient determined based on the health status.

5. The mobile computing device of claim 3, wherein the external device comprises a secured external data store accessible to a physician associated with the patient, a caregiver associated with the patient, or a healthcare expert system.

6. The mobile computing device of claim 1, wherein the processor executes the computer-executable instructions to store at least one of the biometric data, information related to the health status, and information related to the therapeutic feedback in a data store,
wherein the data store is at least one of internal to the mobile computing device or external to the mobile computing device.

7. The mobile computing device of claim 6, wherein the processor executes the computer-executable instructions to:
receive an input from the patient related to at least one of an activity, symptoms, status, a medication, a body position and a food consumption; and
store information related to the input in the data store.

8. A system for smart mobile health monitoring, comprising:
a sensor coupled to a patient and configured to detect biometric data associated with the patient; and
a mobile computing device, comprising:
a memory that stores computer-executable instructions; and
a processor that executes the computer-executable instructions to at least:
receive the biometric data from the sensor;
process the biometric data to at least one of monitor a health status of the patient, diagnose a medical condition of the patient, or diagnose a disease of the patient; and
provide therapeutic feedback related to the health status and at least one of an activity of the patient and a body position of the patient.

9. The system of claim 8, wherein the processor executes the computer-executable instructions to transmit at least one of the biometric data, data derived from the biometric data, information about the health status, and information about the therapeutic feedback to an external device via a wireless protocol.

10. The system of claim 9, wherein the wireless protocol comprises a Bluetooth protocol, a Bluetooth low energy protocol, a ZigBee protocol, an ANT+ protocol, and a WiFi protocol.

11. The system of claim 9, wherein the external device is a therapeutic device configured to deliver a therapeutic treatment to the patient based on the at least one of the biometric data, information about the health status, and information about the therapeutic feedback.

12. The system of claim 8, wherein the mobile computing device is configured to display at least one of the biometric data, data derived from the biometric data, information about the health status, and information about the therapeutic feedback.

13. The system of claim 8, wherein the sensor is coupled to the mobile computing device according to a wired connection comprising a plurality of electrodes implemented in a platform that, upon placement at an appropriate location on the patient, is configured to maintain each of the set of electrodes in an anatomically correct position without placement of individual leads.

14. The system of claim 8, wherein the sensor is at least one of remotely configured, dynamically configured, and adapted for a specific physiological state, a condition, or a specific disease to provide optimized feedback and diagnostic capabilities.

15. The system of claim 8, wherein the mobile computing device configures a global positioning system (GPS) device configured to determine a first location of the mobile computing device; and
wherein the processor executes the computer-executable instructions to select an external device to receive a transmission at least one of the biometric data, information about the health status, and information about the therapeutic feedback to a remote device based on the first location of the mobile device and a second location of the external device.

16. The system of claim 8, wherein the processor executes the computer-executable instructions to, when indicated by the health status exceeding a threshold, communicating with an external electrical stimulator to provide at least one of electrical, chemical, and drug induced stimuli to the patient.

17. A non-transitory computer-readable device storing instructions executable by an associated processor to perform operations that facilitate smart mobile health monitoring, the operations comprising:
receiving biometric data detected by a sensor coupled to a body of a patient;
processing the biometric data to determine a health status of the patient;
providing therapeutic feedback related to the health status; and
transmitting at least one of the biometric data, information related to the health status, and information related to the therapeutic feedback to an external device according to a wireless protocol.

18. The non-transitory computer-readable device of claim 17, wherein the operations further comprise receiving an input from the external device comprising at least one of a query, processed data, an instruction to adjust at least one of the health status and the therapeutic feedback for diagnostic purposes, and an instruction to change patient treatment regiments.

19. The non-transitory computer-readable device of claim 17, wherein the external device is configured to provide a therapeutic procedure to the patient based on at least one of the biometric data, information related to the health status, and information related to the therapeutic feedback

20. The non-transitory computer-readable device of claim 17, wherein the biometric data is related to at least one of an electrocardiogram (ECG), skin potential (EDA), electroencephalogram (EEG), electromyogram (EMG), a heart rate, body impedance, a fluid status, a respiration, a cardiac output, a fall, an activity, a body position, a pulse wave form, blood oxygen levels, a plethysmograph signal, venous/arterial blood pressure waveform, diastolic blood pressure, and a systolic blood pressure.
